(19) United States
(12) Patent Application Publication
(10) Pub. No.: US 2025/0214938 A1
(43) Pub. Date: Jul. 3, 2025

(54) PROCESS FOR PREPARING ((1S,2S)-2-(5-METHYLPYRIDIN-2-YL)CYCLOPROPYL)-METHANOL

(71) Applicants: Merck Sharp & Dohme LLC, Rahway, NJ (US); Organon Pharma (UK) Limited, Greater London (GB)

(72) Inventors: John Y. L. Chung, Edison, NJ (US); Gao Shang, Doylestown, PA (US); David R. Snead, Santee, CA (US); Lushi Tan, Edison, NJ (US); Francois Levesque, Morris Plains, NJ (US); Andrew W. Gibson, Hertfordshire (GB); David R. Lieberman, Greater London (GB); Chengqian Xiao, Shanghai (CN); Xiaxia Zhao, Shanghai (CN); Zhenbing Zhao, Shanghai (CN); Donglin Li, Shanghai (CN)

(73) Assignees: Merck Sharp & Dohme LLC, Rahway, NJ (US); Organon Pharma (UK) Limited, Greater London (GB)

(21) Appl. No.: 18/851,250

(22) PCT Filed: Mar. 27, 2023
(86) PCT No.: PCT/US2023/016411

§ 371 Date: Sep. 26, 2024

(30) Foreign Application Priority Data
Apr. 1, 2022 (WO) PCT/CN2022/084820

(51) Int. Cl. C07D 213/30; C07D 405/06; C07F 1/02

(52) U.S. Cl. CPC C07D 213/30; C07D 405/06; C07F 1/02

(57) ABSTRACT

The present invention relates to an efficient scalable synthesis of compounds such as (1S,2S)-2-(5-methylpyridin-2-yl)cyclopropyl)methanol, which contains a disubstituted cyclopropane with two stereogenic centers and represents a significant challenging synthetic target.

### BACKGROUND OF THE INVENTION

The present invention relates to an efficient scalable synthesis of ((1S,2S)-2-(5-methylpyridin-2-yl)cyclo-propyl) methanol (Compound 5). Compound 5 contains a disubstituted cyclopropane with two stereogenic centers which represents a significant challenging synthetic target. WO2013/028590 discloses a 5-step synthetic route to Compound 5. See also Mordini, A.: et al. Tetrahedron. 2005, 61, 3349-3360. Although this route is able to generate kilogram quantities of Compound 5, it is not suitable for large scale manufacturing due to: (1) potential safety/explosion hazard of ethyl diazoacetate at elevated reaction temperature (135° C.); (2) lack of diastereo- and enantio-control for the cyclopropanation: (3) low efficiency in silica and SFC chromatographies to remove undesired stereoisomers; and (4) poor overall yield (15%).

Therefore, a more efficient and safe synthesis that avoids or improves these issues is desired.

### SUMMARY OF THE INVENTION

A safe and efficient process for making compounds of formula 5′, such as Compound 5, both depicted below, featuring a selective lithiation of 2,5-lutidine, a regioselective addition of lutidyllithium to (S)-epichlorohydrin, a salt metathesis-accelerated epoxide formation, and a trans-selective intramolecular cyclopropanation

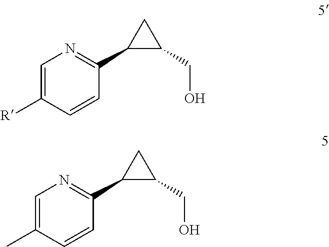

is described.

Further provided is a process for making compounds such as Compound 5 that reduces or eliminates the explosion hazard associated with use of ethyl diazoacetate. Further provided is a process for making compounds such as Compound 5 wherein control of diastereomeric excess (de) and enantioselectivity excess (ee) is achieved. Further provided is a process wherein the chiral purity of compounds such as Compound 5 can be achieved in >99.5% de and >99.5% ee. Further provided is a process that results in compounds such as Compound 5 with less than 0.25% cis isomers detected after crystallization. Further provided is a process for making compounds such as Compound 5 that requires no chromatographic purification. Further provided is a process for making compounds such as Compound 5 with a yield of at least 50%. Further provided is a process for making compounds such as Compound 5 in kilogram quantities with an isolated yield of at least 50% and purity of 99.9% LCAP (liquid chromatography area percent) and 99.9% ee using a continuous flow process. Further provided is a process for making compounds such as Compound 5 in kilogram quantities with an isolated yield greater than 50% and purity of 99.9% LCAP and 99.9% ee using a continuous flow process. Further provided is a process for making Compound 5 within about 100 hours. This and other aspects of the invention are realized upon review of the entire specification.

### BRIEF DESCRIPTION OF THE DRAWINGS

FIG. 1: Depicts the effect of co-solvent agents TMEDA and DBU on the trans/cis ratio of 5.

FIG. 2: Depicts the effect of strong bases nBuLi vs LDA in Step 4 on the yield of 5 and levels of dimer impurity.

### DETAILED DESCRIPTION OF THE INVENTION

The presence of two stereogenic centers on the disubstituted cyclopropane ((1S,2S)-2-(5-methylpyridin-2-yl)cyclo-propyl) methanol (Compound 5) makes it a significantly challenging synthetic target. Provided is a process for making a compound of formula 5′

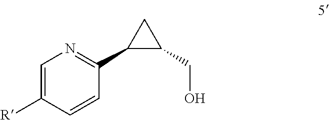

comprising the steps of
1) mixing a compound of formula 1′:

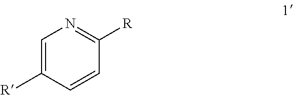

wherein R is selected from C₁₋₁₀ alkyl, C₁₋₆ alkylOR\hat{ }, C₁₋₃haloalkyl, (CH₂)ₙC₄₋₁₀heterocyclyl, (CH₂)ₙC₃₋₁₀cycloalkyl, (CH₂)ₙC₆₋₁₀aryl and (CH₂)ₙC₅₋₁₀heteroaryl; R′ is selected from hydrogen, C₁₋₁₀ alkyl, C₁₋₆ alkylOR, C₁₋₃ haloalkyl, —(CH₂)ₙC₄₋₁₀heterocyclyl, —(CH₂)ₙC₃₋₁₀cycloalkyl, —(CH₂)ₙC₆₋₁₀aryl, and —(CH₂)ₙC₅₋₁₀heteroaryl, and R\hat{ } is hydrogen or C₁₋₆ alkyl, and n is 0 to 3; with a first strong base at a temperature of about 0° C. to about −70° C. to produce a compound of formula 2′,

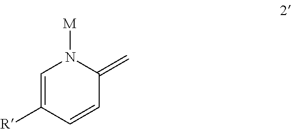

wherein M is selected from lithium (Li), sodium (Na), potassium (K),
2) treating the compound of formula 2′ with a (S)-epichlorohydrin at a temperature of about −100° C. to about 0° C., to produce a compound of formula 3′,

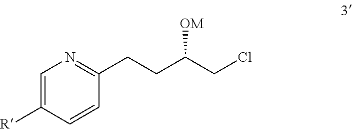

wherein M is selected from lithium, sodium and potassium,
3) aging solution containing the compound of formula 3′ to produce the compound of formula 4′,

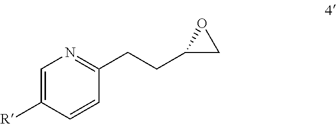

4) treating the compound of formula 4′ with a second strong base to produce the compound of formula 5′ and isolating the compound of formula 5′.

An embodiment is realized when the process for making a compound of formula 5′ is run in a batch mode. An embodiment is realized when the process for making a compound of formula 5′ is run in a continuous flow mode. A subembodiment of the process is realized when the continuous flow mode is selected from PFR (plug flow reactor) design or CSTR (continuous stirred tank reactor) design, or combination thereof. Another subembodiment of the process is realized when the continuous flow mode is PFR. Another subembodiment of the process is realized when the continuous flow mode is CSTR. Another subembodiment of the process is realized when the continuous flow mode uses a combination of PFR and CSTR modes. Still another subembodiment of the process is realized when Steps 1 and 2 are run using PFR mode and Steps 3 and 4 are run using CSTR mode.

Another embodiment of the process is realized when the first and second strong base is selected from the group consisting of methyllithium, ethyllithium, butyl lithium (nBuLi, sec-BuLi, t-BuLi), hexyl lithium (nHexLi), cyclohexyllithium, lithium diisopropylamide (LDA), BuLi+tert-BuOK, lithium hexamethyldisilazide (LiHMDS), sodium bis(trimethylsilyl)amide (NaHMDS), potassium bis(trimethylsilyl)amide (KHMDS), lithium diethylamide (LDEA), sodium amide (NaNH₂), sodium hydride (NaH), lithium tetramethylpiperidine, tetramethylpiperidine sodium, and tetramethylpiperidine potassium, wherein, depending on the first strong base used, M is a cation selected from sodium, potassium, or lithium.

A subembodiment of this process is realized when the first strong base is selected from methyllithium, ethyllithium, butyl lithium (nBuLi, sec-BuLi, t-BuLi), hexyl lithium (nHexLi), cyclohexanyllithium, lithium diisopropylamide (LDA), and BuLi+tert-BuOK. Another subembodiment of this aspect of the invention is realized when the first strong base is selected from nBuLi, sec-BuLi, t-BuLi, hexyl lithium (nHexLi), and lithium diisopropylamide (LDA). Another subembodiment of this aspect of the invention is realized when the first strong base is selected from nBuLi, sec-BuLi, t-BuLi, and hexyl lithium (nHexLi).

Another subembodiment of this aspect process is realized when the second strong base is selected from nBuLi, sec-BuLi, t-BuLi, hexyl lithium (nHexLi), and lithium diisopropylamide (LDA), or mixture thereof. Another subembodiment of this aspect of the invention is realized when the second strong base is selected from nBuLi, hexyl lithium (nHexLi) and lithium diisopropylamide (LDA) or mixture thereof. Another subembodiment of this aspect of the invention is realized when the second strong base is nBuLi. Another subembodiment of this aspect of the invention is realized when the second strong base is hexyl lithium (nHexLi). Another subembodiment of this aspect of the invention is realized when the second strong base is lithium diisopropylamide (LDA).

Another embodiment of this process is realized when the ratio of the compound of formula 1′ to first strong base is about 3:1, 2:1, 1.5:1, 1.25:1, 1:1, 1:1.5, or 1:0.5, respectively. A subembodiment of this aspect of the invention is realized when the ratio of the compound of formula 1′ to first strong base is 1:1, respectively.

Another embodiment of this process is realized when Step 1 is conducted at a temperature of about −10° C. to about −50° C., −15° C. to about −45° C., −20° C. to about −40° C., or −20° C. to about −30° C. A subembodiment of this aspect of the process is realized when Step 1 is conducted at a temperature of about −20° C. to about −30° C.

Another embodiment of this process is realized when R is selected from hydrogen, C₁₋₁₀ alkyl, C₁₋₆ alkylOR\hat{ }, and C₁₋₃haloalkyl.

Another embodiment of this process is realized when R′ is selected from C₁₋₁₀ alkyl, C₁₋₆ alkylOR, and C₁₋₃ haloalkyl.

In a further embodiment, R′ is methyl.

Another embodiment of this process is realized when n is 1. Another embodiment of this process is realized when n is 2. Another embodiment of this process is realized when n is to 3.

Another embodiment of this process is realized when the ratio of the compound of formula 2′ to (S)-epichlorohydrin in Step 2 is about 2:1, 1.5:1, 1.25:1, 1:1, 1.0:1.1, 1:1.3, 1:1.5, or 1:0.5, respectively. A subembodiment of this aspect of the process is realized when the ratio of the compound of formula 2′ to (S)-epihalohydrin is 1:1.1 to 1.5, respectively.

Another embodiment of this process is realized when Step 2 is conducted at a temperature of about −90° C. to about −50° C., −80° C. to about −65° C., −80° C. to about −55° C. or −78° C. to about −60° C. A subembodiment of this aspect of the process is realized when Step 2 is conducted at a temperature of about −78° C. to about −60° C.

When M is lithium in compound of formula 3″, it's conversion to compound of formula 4′ in Step 3 is aided by the addition of an alkoxide reagent or a co-solvent or mixture thereof. An embodiment of this process is realized when M is lithium. A subembodiment of this aspect of the process is realized when a alkoxide reagent is added in Step 3 when M is lithium. A subembodiment of this aspect of the process is realized when the alkoxide reagent and/or co-solvent is selected from NaOtBu, KOtBu. DMPU, HMPA or mixture thereof. A subembodiment of this aspect of the process is realized when the alkoxide reagent is NaOtBu. Another subembodiment of this aspect of the process is realized when the alkoxide reagent is KOtBu. Another subembodiment of this aspect of the process is realized when the co-solvent is DMPU. Another subembodiment of this aspect of the process is realized when the co-solvent is HMPA. Another subembodiment of this aspect of the process is realized when the alkoxide reagent is a mixture of NaOtBu and KOtBu. Another embodiment of this aspect of the process is realized when the ratio of the compound of formula 3′ to alkoxide reagent is about 1:3, 1:2.5, 1:2.2, 1:2.0, or 1:1.5, respectively. A subembodiment of this aspect of the process is realized when the ratio of the compound of formula 3′ to alkoxide reagent is 1:2.2, respectively.

To improve yield and stereoselectivity a bidentate ligand (also referred to as deaggregation aid) optionally is added in Step 3 or Step 4. Another embodiment of this aspect of the process is realized when a bidentate ligand aid optionally is added in Step 3 or Step 4 when M is lithium A subembodiment of this aspect of the process is realized when the bidentate ligand is selected from diazabicycloundecene (DBU), 1,2-dimethoxyethane, and tetramethylethylene diamine, or mixture thereof. A subembodiment of this aspect of the process is realized when the bidentate ligand added in Step 3 or Step 4 is diazabicycloundecene (DBU). A subembodiment of this aspect of the process is realized when the bidentate ligand add in Step 3 or Step 4 is 1,2-dimethoxyethane. A subembodiment of this aspect of the process is realized when the bidentate ligand added in Step 3 or Step 4 is tetramethylethylene diamine. A subembodiment of this aspect of the process is realized when the ratio of the compound of formula 3′ to bidentate ligand is about 1:4, 1:3, or 1:2.5, respectively. A subembodiment of this aspect of the process is realized when the ratio of the compound of formula 3′ to bidentate ligand aid is 1:3, respectively. A subembodiment of this aspect of the process is realized when the addition of the bidentate ligand gives an additional increase in trans:cis ratio. A further subembodiment of this aspect of the process is realized when the addition of the bidentate ligand gives an additional increase in trans:cis ratio from about 6:1 up to about 16:1.

A subembodiment of this process is realized when an alkoxidereagent and bidentate ligand are both added in Step 3 when M is lithium to produce the compound of the compound of formula 4′. A subembodiment of this aspect of the process is realized when the ratio of the compound of the compound of formula 3′ to alkoxide reagent to bidentate ligand is about 1:3:4, 1:2.5:4, 1:2.5:3, 1:2.2:3, 1:2.0:3, or 1:1.5:2.5, respectively. A subembodiment of this aspect of the process is realized when the ratio of the compound of formula 3′ to chelating agent to bidentate ligand is 1:2.2:3, respectively.

Another embodiment is realized by a process wherein about 90% average yield is obtained with each of steps 1, 2 and 3.

Another embodiment of this process is realized when Step 3 is conducted at a temperature of about −50° C. to about 5° C., −20° C. to 0° C., or −10° C. to about 0° C. A subembodiment of this aspect of the process is realized when Step 3 is conducted at a temperature of about −10° C. to about 0 A subembodiment of this process is realized when the second strong base, lithium diisopropylamide (LDA) in Step 4, results in a compound of formula 5′ total yield of >50%. A subembodiment of this aspect of the process is realized when formation of the compound of formula 5′ occurs in about 90:10 trans:cis ratio. Another subembodiment of this aspect of the invention is realized when the yield of the compound of formula 5′ in Step 4 is 50% to 70%.

Another embodiment of this process is realized when the mixture comprising the compound of formula 5′ produced in Step 4 is optionally aged at about −25° C. to about 0° C. for about 0 to 72 hours, or about 24 to 48 hours to produce a compound of formula 5′ with a trans/cis ratio of 96:4.

As used herein, “alkyl” refers to both branched- and straight-chain saturated aliphatic hydrocarbon groups having the specified number of carbon atoms in a specified range. For example the term “C₁₋₁₀ alkyl” means linear or branched chain alkyl groups, including all possible isomers, having 1, 2, 3, 4, 5, 7, 8, 9 or 10 carbon atoms, and includes each of the decyl, nonyl, octyl, heptyl, hexyl and pentyl isomers as well as n-, iso-, sec- and tert-butyl (butyl, s-butyl, i-butyl, t-butyl; Bu=butyl, collectively “—C₄alkyl”), n- and iso-propyl (propyl, i-propyl, Pr=propyl, collectively “—C₃alkyl”), ethyl (Et) and methyl (Me). “C₁₋₄alkyl” has 1, 2, 3 or 4 carbon atoms, and includes each of n-, iso-, sec- and tert-butyl, n- and i-propyl, ethyl and methyl.

The term “cycloalkyl” as employed herein includes saturated cyclic, bicyclic, tricyclic, or polycyclic hydrocarbon groups having 3 to 12 carbons. The cycloalkyl groups herein described may also contain fused rings. Fused rings are rings that share a common carbon-carbon bond or a common carbon atom (e.g., spiro-fused rings). Examples of cycloalkyl moieties include, but are not limited to, cycylopropyl, cyclobutyl, cyclopentyl, cyclohexyl, adamantyl, norbornyl, and decalin.

As used herein, “aryl” refers to (i) phenyl, (ii) 9- or 10-membered bicyclic, fused carbocylic ring systems in which at least one ring is aromatic, and (iii) 11- to 14-membered tricyclic, fused carbocyclic ring systems in which at least one ring is aromatic. Suitable aryls include, for example, substituted and unsubstituted phenyl and substituted and unsubstituted naphthyl. An aryl of particular interest is unsubstituted or substituted phenyl.

The term “heterocyclyl” refers to a nonaromatic 3-10 membered monocyclic, 8-12 membered bicyclic, or 11-14 membered tricyclic ring system having 1-3 heteroatoms of monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heterocyclyl groups herein described may also contain fused rings. Fused rings are rings that share a common carbon-carbon bond or a common carbon atom (e.g., spiro-fused rings). Examples of heterocyclyl include, but are not limited to tetrahydrofuranyl, tetrahydropyranyl, piperidinyl, morpholino, pyrrolinyl and pyrrolidinyl.

The term “heteroaryl” refers to an aromatic 5-8 membered monocyclic, 8-12 membered bicyclic, or 11-14 membered tricyclic ring system having 1-3 heteroatoms for monocyclic. 1-6 heteroatoms for bicyclic, or 1-9 heteroatoms for tricyclic, said heteroatoms selected from O, N, or S (e.g., carbon atoms and 1-3, 1-6, or 1-9 heteroatoms of N, O, or S for monocyclic, bicyclic, or tricyclic, respectively). The heteroaryl groups herein described may also contain fused rings that share a common carbon-carbon bond.

### EXAMPLES

### General Methods

Solvents, reagents, and intermediates that are commercially available were used as received. Reagents and intermediates that are not commercially available were prepared in the manner as described herein. 1H NMR spectra are reported as ppm downfield from Me₄Si with number of protons, multiplicities, and coupling constants in Hertz indicated parenthetically. Examples of organic solvents useful for this invention are tetrahydrofuran (THF), 2-methyl-tetrahydrofuran (2-MeTHF), (methyl tert-butyl ether) MTBE, ethanol, propanol, isopropanol, acetonitrile, acetone, heptane, hexane, toluene, methanol, or mixtures thereof.

The following batch (Example 1) and continuous flow (Example 2) examples depict a process which begins with lithiation of 2,5-picoline with n-BuLi or n-HexLi to generate the lithiated picoline 1a. Subsequent treatment with (S)-epichlorohydrin led to formation of chlorohydrine lithium salt 3. This reaction was highly exothermic and therefore was kept cold to minimize the formation of bis-adduct and bicyclic pyridinium impurities. Intermediate 3 when M was lithium was slow to convert to epoxide 4, therefore a salt metathesis to the sodium salt was carried out to accelerate the epoxide formation. This was accomplished by addition of sodium tert-butoxide which drove the epoxidation by precipitation of NaCl.

After complete formation of 4, it was treated with a base n-HexLi, or LDA to generate the internal picolyl anion which underwent an intramolecular displacement of the epoxide to afford cyclopropane 5 with >97% ee (Example 1). Formation of trans-isomer of 5 was favored in typically 90:10 trans:cis ratio. Reaction yields of about 55 to about 70% was obtained at scales ranging from about 10 g-15 kg in batch modes (Example 1). Aging this mixture at −20° C. for 12 or more hours gave an improved trans/cis ratio of 96:4 in batch mode.

With continuous flow mode (Example 2) about 70% reaction yield was achieved over 4-steps in a continuous flow process, with the first 3 steps executed with a reaction yield of near 90%. Initial reaction yield in the fourth step was below 55%. To increase the yield, bidentate ligands selected from 1,2-dimethoxyethane, tetramethylethylene diamine and diazabicycloundecene (DBU) optionally can be added in Step 3 or Step 4.

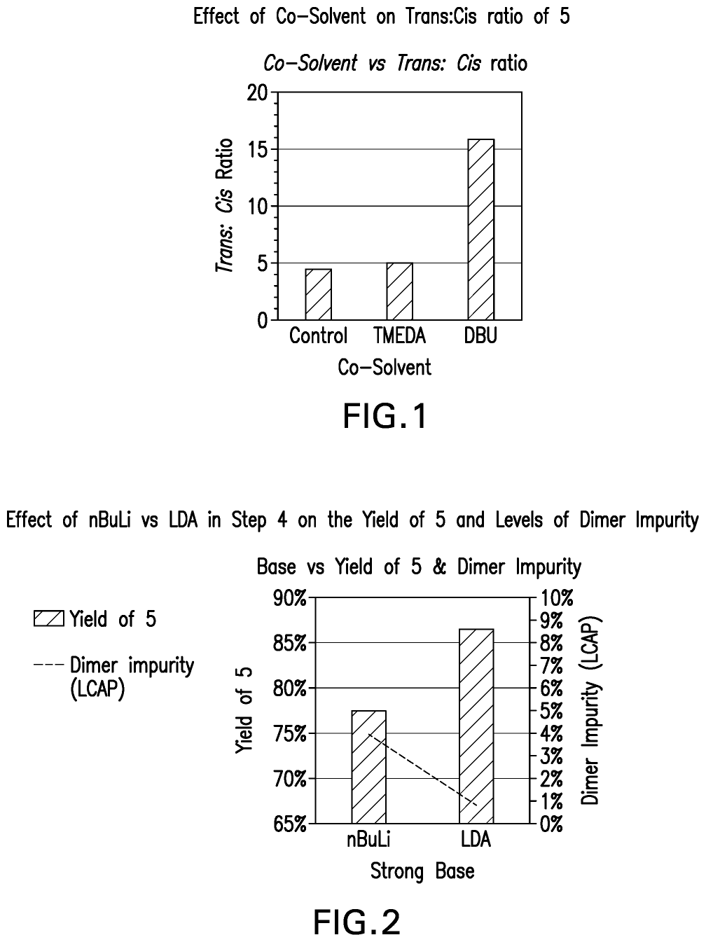

Diazabicycloundecene (DBU) was added in Step 3 of Example 2, which improved the trans:cis ratio from 6:1 up to about 16:1. Use of LDA in Step 4 suppressed the hard-to-purge dimer impurity (compound Z) from about 4% to about 1%, reduced impurity level, improved the trans:cis ratio and further increased the yield by about 10%. (See FIG. 1). Through this process with the addition of DBU and LDA, the reaction yield was increased by about 20%, with gain in selectivity as well. Example 2 illustrates a scaled-up run in continuous flow mode producing about 30 kg of 5 in ˜100 h, with 52% isolated yield, 99.9% LCAP, 99.9% ee, avg. 85%/step. Compound Z: based on Mass Spectrometer data without further structure elucidation work

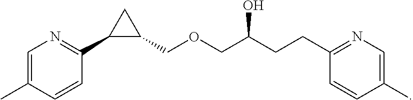

### Example 1

### Preparation of Picolyl-Cyclopropylmethanol-Batch Mode (15 kg Scale)

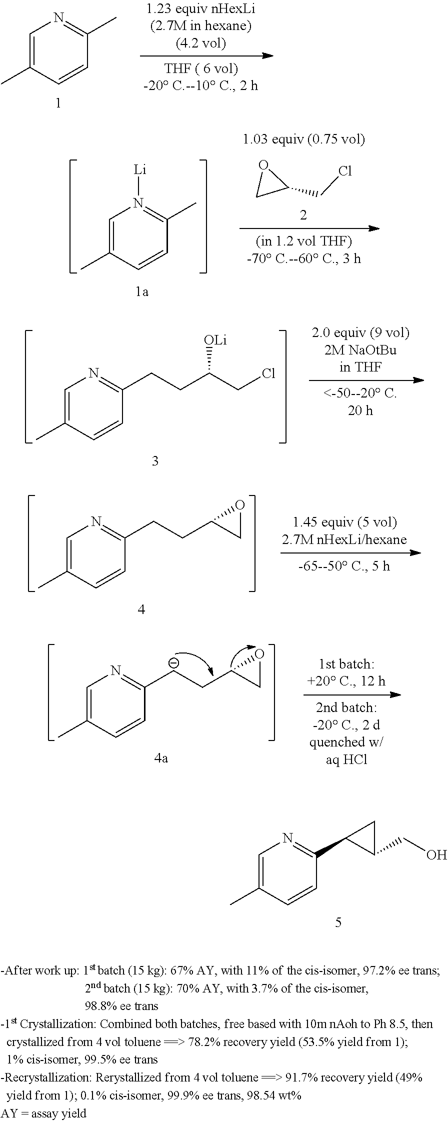

### Example 2

### Synthesis of (S,S)-2-(5-methylpyridin-2-yl)cyclopropyl methanol-continuous mode

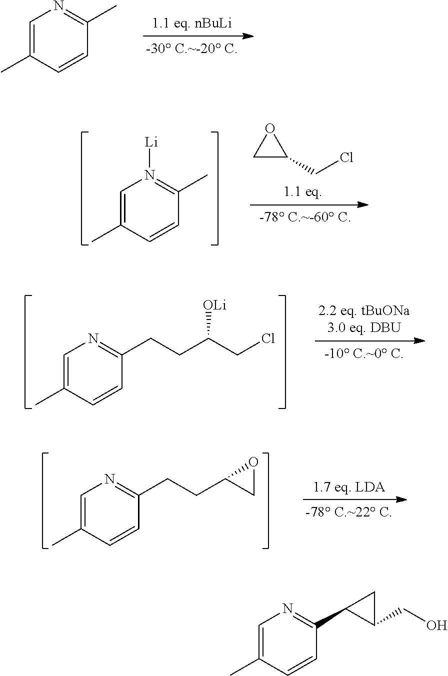

### Experimental Procedure

Below details a production run starting from 31 kg of 2,5-lutidine providing 27.5 kg of desired product dry cake with 99.2% purity in 56.4% yield.

### Preparation of Solution A:

2,5-lutidine (31.6 kg, 294.87 mol) and anhydrous 2-MeTHF (568.1 kg or 660.6 L, Karl Fischer (KF) 0.02% were combined with overhead stirring, under N₂ atmosphere. The resulting solution KF was 0.04%, meeting the target of <0.05%. This 2,5-lutidine solution (0.42 M) was then transferred into a drum A via a filter.

### Preparation of Solution B:

nBuLi in hexanes (87.3 kg, 126 L, 314.9 mol, 2.5 M) was maintained under N₂ atmosphere in a drum B and used as is from a commercial supplier.

### Preparation of Solution C:

(S)-(+)-epichlorohydrin (34.2 kg, 369.67 mol) was combined with anhydrous 2-MeTHF (265.1 kg or 308.3 L, KF 0.02%). The resulting solution KF was 0.04% and met the target of <0.05%. This solution (1.09 M) was transferred into a drum C via a filter.

### Preparation of Solution D:

BuONa (71.5 kg), DBU (148.6 kg) and anhydrous THF (664 kg or 746 L kg. KF 0.03%) were combined under N₂ atmosphere. The resulting solution (0.8 M/BuONa) was transferred into a drum D via a filter.

### Preparation of Solution E:

LDA in THF/nheptane (12:25, v/v. 224 kg, 2.0 M) was charged to a drum E.

### Preparation of Solution F:

HCl (864 kg, 3.4 M) was charged to a drum F via a filter

### Procedure:

Solution A (0.42 M, 112.2 mL/min) was combined with Solution B (2.5 M, 20.6 mL/min) for 10 min at −30˜ 10° C. (wherein ˜ represents cooled to about −30° C. to begin then allowed it to exotherm to upper limit of about −10° C. as reaction proceeded), in a reactor 1 (PFR (1.4 L). Solution AB was then combined with Solution C (1.09 M, 47.5 mL/min) for 15 min at <−60° C., in a reactor 2 (PFR (2.7 L). Solution ABC was then combined with Solution D (0.8 M, 145 mL/min) for 5 min at −30˜−10° C., in a reactor 3A (CSTR 1.6 L). The reaction stream was then transferred to a reactor 3B (CSTR 3.2 L) for 10 min at −5˜10° C., then to a reactor 3C (CSTR 3.2 L) for 10 min at 0˜10° C., and finally to a reactor 3D (CSTR 1.6 L) for 5 min at <−30° C. Solution ABCD was then combined with mixture of Solution E (2.0 M, 39.8 mL/min) and THF solution (39.8 mL/min) for 5 min at <−30° C. in Reaction 4A (CSTR 2.0 L). The reaction stream was then transferred to reactor 4B (CSTR 4.3 L) for 10 min at 0˜25° C., and finally reactor 4C (CSTR 83.0 L) for 210 min at 15˜25° C. Solution ABCDE was then combined with quench Solution F (3.4 M, 118 mL/min) for 10 min at 0˜15° C. in reactor 5 (CSTR (5.1 L).

The quenched reaction mixture (3129.65 kg) was transferred into reactor R5 in batches. The batch temperature was adjusted to 0-10° C., and adjusted the pH to 8-9 by adding dropwise 30% aq. NaOH or 2 N HCl at 0-10° C. After standing for 0.5-1 hours, the layers were separated. The aqueous layer was back-extracted twice with 2-MeTHF (2×363 kg). The combined organic was concentrated to 8-10 vol. (relative to 2,5-lutidine) at <45° C., then washed with 10 vol 10% aq. Na₂SO₄ (154 kg). The organic layer was analyzed by HPLC showing an 74.4% yield of the desired product (283.5 kg×12.4 wt %=36.15 kg; purity=81.9 LCAP (liquid chromatography area percent), d.r.=94.3:5.7). The organic layer was concentrated to 1-3 vol at <45° C. diluted with 5 vol 2-MeTHF (153 kg), concentrated again to 1-3 vol, then diluted with 5 vol 2-MeTHF (232 kg). If KF of the solution is <1% proceeds to the next step, otherwise, repeat the flushing. The organic solution was solvent-switched to toluene by concentrating to 1-2 vol at <45° C., diluting with toluene (152 kg) and concentrated until 1-2 vol at <60° C. This was repeated with 1×152 kg and 1×171 kg toluene and then concentrated to 4-5 vol. The temperature was adjusted to 55-65° C. and stirred until the solution was clear. The solution was slowly cooled to 35-45° C. over 3-5 hours. Sample was taken to determine residual THF and 2-MeTHF (THF: not detected: 2-MeTHF <0.01%). The solution was seeded with (S,S)-2-(5-methylpyridin-2-yl)cyclopropyl methanol (˜100 g), and continued to stir at 35-45° C. for 2-5 h. The temperature was then adjusted to 0-10° C. and stirred for 3-8 h at which point a sample was taken showing loss of desired product to supernatant was ˜5% and solid purity=99.4% with d.r.=99.9:0.1). The batch was filtered, washed with cold toluene (60 kg), then vacuum dried at 45-50° C. for 10-20 h affording 27.5 kg (S,S)-2-(5-methylpyridin-2-yl)cyclopropyl methanol in 56.4% isolated yield (purity 96.8 wt %, 99.2 LCAP, d.r. 99.9:0.1, e.r. 99.3:0.7).

The purity of product was further upgraded by recrystallization from acetone/water to afford the desired product in 100% purity with e.r. 99.92:0.08. Starting from 32.3 kg crude product (27.55 kg×96.8 wt %+3.17 kg×99.9 wt %+2.16 kg×97.8 wt %+0.36 kg×97.8 wt %), which after the recrystallization and drying produced 29.54 kg cake in 90.8% recovery yield. Based on 31 kg 2,5-lutidine, the isolated yield was 51.2%. Detailed procedure is as follows: To reactor RI was charged 32.3 kg (assayed) crude product, acetone (26 kg) and H₂O (172 kg). The mixture was stirred at 50-60° C. for 0.5-3.5 h until turned clear. The batch temperature was adjusted to 35-45° C., and then seeded with 0.21 kg desired product. After stirring for 0.5-5 h at 35-45° C., the mixture was concentrated under reduced pressure to 126-158 L at <35° C. The batch was cooled to 0-10° C. over 1-3 h, aged at this temperature for 5-10 h, then filtered, and the wet cake was washed with cold H₂O (49 kg). Sample was taken to test the purity and chiral purity, purity: 100.0%, chiral purity: 0.08%/99.92%. The wet cake was dried under vacuum at 40-50° C. for 10-20 h, affording 29.5 kg of target compound. Samples were taken to test the residual solvents in dry cake: toluene <100 ppm, THF <100 ppm, 2-MeTHF <100 ppm, n-hexane <100 ppm, n-heptane <100 ppm, acetone=332 ppm, ethylbenzene <100 ppm, DBU=8 ppm, DIPA <100 ppm, KF=0.2%.

1H NMR (400 MHZ, Methanol-d₄) δ 0.89-1.02 (m, 1H) 1.15 (dt, J=8.78, 4.64 Hz, 1H) 1.53-1.64 (m, 1H) 1.98 (dt, J=8.72, 4.55 Hz, 1H) 2.29 (s, 3H) 3.45-3.55 (m, 1H) 3.59-3.70 (m, 1H) 7.13 (d, J=8.03 Hz, 1H) 7.50 (br d, J=7.28 Hz, 1H) 8.19 (s, 1H). 13C NMR (101 MHz, Methanol-d₄) § 12.97 (s, 1C), 16.55 (s, 1C), 21.95 (s, 1C), 24.94 (s, 1C), 64.57 (s, 1C), 120.71 (s, 1C), 130.31 (s, 1C), 137.32 (s, 1C), 148.22 (s, 1C), 158.83 (s, 1C). Chiral purity 99.84% ee; 100% de. HRMS (ESI) m/z calcd. for C₁₀H₁₄NO [M+H]⁺ 164.1070, found 164.1070.

1. A process for making a compound of formula 5′ 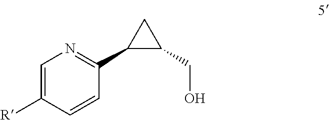
comprising the steps of
1) mixing a compound of the compound of formula 1′: 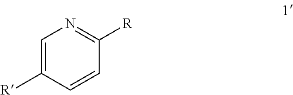
wherein R is selected from C₁₋₁₀ alkyl, C₁₋₆ alkylOR\hat{ }, C₁₋₃haloalkyl, (CH₂)ₙC₄₋₁₀heterocyclyl, (CH₂)ₙC₃₋₁₀cycloalkyl, (CH₂)ₙC₆₋₁₀aryl and (CH₂)ₙC₅₋₁₀heteroaryl; R′ is selected from hydrogen, C₁₋₁₀ alkyl, C₁₋₆ alkylOR, C₁₋₃ haloalkyl, —(CH₂)ₙC₄₋₁₀heterocyclyl, —(CH₂)ₙC₃₋₁₀cycloalkyl, —(CH₂)ₙC₆₋₁₀aryl, and —(CH₂)ₙC₅₋₁₀heteroaryl, and R\hat{ } is hydrogen or C₁₋₆ alkyl, and n is 0 to 3;
with a first strong base at a temperature of about 0° C. to about −70° C. to produce a compound of formula 2′, 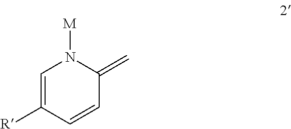
wherein M is selected from lithium (Li), sodium (Na), and potassium (K),
2) treating the compound of formula 2′ with a (S)-epichlorohydrin at a temperature of about −100° C. to about 0° C., to produce a compound of formula 3′, 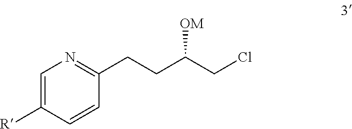
wherein M is selected from lithium, sodium and potassium,
3) aging solution containing the compound of formula 3′ to produce the compound of formula 4′, 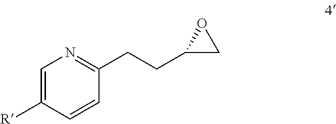
4) treating the compound of formula 4′ with a second strong base to produce the compound of formula 5′ and isolating the compound of structural formula 5′.

2. The process according to claim 1 wherein the first strong base is selected from the group consisting of methyllithium, ethyllithium, n-butyl lithium, sec-butyl lithium, t-butyl lithium, hexyl lithium, cyclohexyllithium, lithium diisopropylamide, BuLi+tert-BuOK, lithium hexamethyldisilazide, sodium bis(trimethylsilyl)amide, potassium bis(trimethylsilyl)amide, lithium diethylamide, sodium amide, sodium hydride, lithium tetramethylpiperidine, tetramethylpiperidine sodium, and tetramethylpiperidine potassium.

3. A process according to claim 1 wherein the first strong base is selected from methyllithium, ethyllithium n-butyl lithium, sec-butyl lithium, t-butyl lithium, n-hexyl lithium, cyclohexyllithium, and lithium diisopropylamide.

4. The process according to claim 1 wherein Step 1 is conducted at a temperature of about −10° C. to about −50° C.

5. The process according to claim 1 wherein Step 2 is conducted at a temperature of about −90° C. to about −50° C.

6. The process according to claim 1 wherein M is lithium, and an alkoxide reagent, or co-solvent or mixture thereof is added in Step 3.

7. The process according to claim 6 wherein the alkoxide reagent and/or co-solvent are selected from NaOtBu, KOtBu, DMPU, and HMPA.

8. The process according to claim 1 wherein M is lithium and a bidentate ligand is added in Step 3.

9. The process according to claim 8 wherein the bidentate ligand selected from diazabicycloundecene, 1,2-dimethoxyethane, and tetramethylethylene diamine.

10. The process according to claim 1 wherein an alkoxide reagent and bidentate ligand is added in Step 3 when M is lithium.

11. The process according to claim 1 wherein Step 3 is conducted at a temperature of about −25° C. to about 5° C.

12. The process according to claim 1 wherein the second strong base is selected from methyllithium, ethyllithium, n-butyl lithium, sec-butyl lithium, t-butyl lithium, hexyl lithium, cyclohexyllithium, lithium diisopropylamide, BuLi+tert-BuOK, lithium hexamethyldisilazide, sodium bis(trimethylsilyl)amide, potassium bis(trimethylsilyl)amide, lithium diethylamide, sodium amide, sodium hydride, lithium tetramethylpiperidine, tetramethylpiperidine sodium, and tetramethylpiperidine potassium or a mixture thereof.

13. The process according to claim 1 wherein the second strong base is selected from n-butyl lithium, sec-butyl lithium, t-butyl lithium, n-hexyl lithium, and lithium diisopropylamide, or a mixture thereof.

14. The process according to claim 1 wherein the second strong base is lithium diisopropylamide.

15. The process according to claim 12 which results in a compound of formula 5′ with >97% ee.

16. The process according to claim 12 wherein the resulting compound of formula 5′ in Step 4 is aged at a temperature of about −25° C. to about 0° C.

17. The process according to claim 16 wherein the resulting compound of formula 5′ has a trans/cis ratio of about 96:4.

18. The process according to claim 1 which is a batch mode process.

19. The process according to claim 18 wherein about 55-70% of a compound of formula 5′ is obtained at scales ranging from 10 g-15 kg.

20. The process according to claim 1 which is a continuous flow process.

21. The process according to claim 20 which gives a yield of a compound of formula 5′ of at least 30 kg.

22. The process according to claim 1 for making a compound of formula 5 [chemical structure]
